(19) United States
(12) Patent Application Publication
(10) Pub. No.: US 2023/0113064 A1
(43) Pub. Date: Apr. 13, 2023

(54) DIAGNOSTIC SYSTEM USING DIFFRACTION ANALYSIS OF IN VITRO SAMPLES

(71) Applicant: Arion Diagnostics, Inc., Petaluma, CA (US)

(72) Inventors: Delvin Tai Wai Yuk, Atherton, CA (US); Alexander P. Lazarev, Lake Forest, CA (US); Pavel I. Lazarev, Menlo Park, CA (US)

(21) Appl. No.: 17/500,766

(22) Filed: Oct. 13, 2021

(51) Int. Cl. G16H 10/40; G16H 50/70; G16H 50/20; G16H 30/00; G06N 3/08

(52) U.S. Cl. CPC G16H 10/40; G16H 50/70; G16H 50/20; G16H 30/00; G06N 3/08

(57) ABSTRACT

Diffractometer-based global in vitro diagnostic systems or methods may use one or more diffraction apparatuses for the structural analysis of α-keratin and collagen in samples of hair, nails, skin, internal organs, or other tissue of a human or non-human animal. The diffraction apparatuses operatively couple to a computer database and provide sample data including diffraction pattern data for in vitro samples or data derived therefrom. One or more computer systems receive or transmit the sample data or data derived therefrom and process the sample data or data derived therefrom to provide a computer-aided diagnostic indicators.

### BACKGROUND

An early diagnosis of malignancies correlates directly with successful treatment of a patient. Yet, patients may present too late for effective treatment for many malignancies because no readily available, noninvasive, cost-effective diagnostic tests exist. In general, diagnosis of malignancies has required direct targeting of affected organs with detection via mammogram, ultrasound, MRI, biopsy, or other methods.

Fiber diffraction techniques have been used in the study of muscle, collagen, and keratin and used to examine changes from normal tissue to pathological tissue specific to a disease of that tissue. P. Lazarev et al., “Human Tissue X-ray Diffraction: Breast, Brain, and Prostate”, Proceedings of the 22nd Annual International Conference of the IEEE Engineering in Medicine and Biology Society, Cat. No. 00CH37143, Vol. 4, pp. 3230-3233, (2000, July) considers the possibility of using small-angle x-ray spectroscopy to study the structure of biological tissues.

Recent research has demonstrated “indirect” detection and diagnostic methods using, for example, hair samples, and specific changes in the structure of hair associated with colon cancer and Alzheimer's disease have been published. Veronica J. James, “Fiber Diffraction of Skin and Nails Provides an Accurate Diagnosis of Malignancies,” Int. J. Cancer: 125, 133-138 (2009) suggests use of fiber diffraction patterns of skin or fingernails, using X-ray sources, as a biometric diagnostic method for detecting neoplastic disorders including but not limited to melanoma, breast, colon, and prostate cancers. The article claims that with suitable further development, an early low-cost, totally noninvasive yet reliable diagnostic test could be conducted on a regular basis in local radiology facilities, as a confirmatory test for other diagnostic procedures or as a mass screening test using suitable small angle X-ray beam-lines at synchrotrons. The article indicates that some human and animal nail samples were examined on the Beach Facility, Advanced Photon Source, and provided excellent diffraction pictures.

Pathological analysis of in vitro tissue samples, e.g., biopsy samples, is an expensive, labor intensive, and time-consuming process which is primarily based on assessment of the visual appearance of the samples. Furthermore, the sample preparation required for visual assessment is also a labor intensive and time-consuming process which may lead to erroneous results in the subsequent analysis. The final analysis of the samples is based on visual assessment by a trained professional, and thus relies on animal judgement and is therefore subjective by nature.

### SUMMARY

Malignancies and other diseases or conditions may leave molecular level “signatures” in hair, skin, and nail tissues as well as in the local tissues that malignancies, diseases, or conditions may directly affect. These distinctive signatures can be distinguished using X-ray fiber diffraction techniques. Biological macromolecules such as collagen and keratin belong to a group of fibrous macromolecules containing long polymeric structures parallel to each other. The α-keratin plates in fingernails are composed of fibers that are intrinsically aligned while orientation of the collagen fibers in the dermal layer of the skin can be achieved by stretching. These pseudo-crystalline rod-like structures can be studied by fiber diffraction. For this, the oriented fibers are placed in a collimated X-ray beam, so that the parallel fibers are at right angles to the beam and the pattern of the X-rays diffracted at very low angles is recorded and analyzed. Diffraction study of such fibers is now routinely achieved in minutes using laboratory-based rotating anodes combined with multilayer optics or suitable small angle scattering synchrotron beams.

The present disclosure describes a sample analysis and communication system that produces an objective diagnostic indicator for subject-sample analyzed by the system, comprising: an subject-sample -analyzer subsystem that includes at least one subject-sample analyzer constructed to analyze animal sample and to produce an objective-diagnostic indicator; and a two-way communication subsystem constructed to allow the subject-sample-analyzer subsystem to send and receive information relevant to the objective-diagnostic indicator.

Disclosed herein are novel methods and systems for fast and accurate in vitro analysis of tissue samples that utilize an objective digital measurement of the structural properties of tissue in the sample, which is directly indicative of its physiological and pathological status.

Another aspect of the present disclosure provides a non-transitory computer readable medium comprising machine executable code that, upon execution by one or more computer processors, implements any of the methods described above or disclosed elsewhere herein.

Another aspect of the present disclosure provides a system comprising one or more computer processors and computer memory coupled thereto. The computer memory comprises machine executable code that, upon execution by the one or more computer processors, implements any of the methods described above or disclosed elsewhere herein.

Additional aspects and advantages of the disclosed concepts will become readily apparent to those skilled in the art upon review of the following detailed description, wherein only illustrative embodiments of the disclosed concepts are shown and described. As will be realized, the concepts of the present disclosure may be implemented in other and different embodiments, and the several details thereof are amenable to modification in various obvious respects, all without departing from the scope of the disclosure. Accordingly, the drawings and description are to be regarded as illustrative in nature, and not as restrictive.

### BRIEF DESCRIPTION OF THE DRAWINGS

FIG. 1 schematically shows the fiber diffraction techniques.

FIG. 2 schematically shows a container for hair samples.

FIG. 3 shows a block diagram illustrating a diagnostic system in accordance with an example of the present disclosure including multiple sample diffractometers operatively coupled to a database over a network.

FIG. 4 shows an example schematic for a data collection and processing workflow.

FIG. 5 shows a computer system that is programmed or otherwise configured to implement methods provided herein.

The drawings illustrate examples for the purpose of explanation and are not of the invention itself. Use of the same reference symbols in different figures indicates similar or identical items.

### DETAILED DESCRIPTION

While various systems and processes have been shown and described herein, it will be obvious to those skilled in the art that such systems and processes are provided by way of example only. Numerous variations, changes, and substitutions may occur to those skilled in the art without departing from the invention. It should be understood that various alternatives to the examples described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Unless otherwise defined, the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the term “samples” generally refers to α-keratin and collagen samples contained in animal hair, nails, skin and biological samples of animal internal organs. These samples can be used for “indirect” detection and diagnosis of various diseases by fiber diffraction.

As used herein, the term “diffraction apparatus” generally refers to an instrument or diffractometer configured to record diffraction data from one or more in vitro tissue samples or specimens. The diffraction apparatus may be an x-ray diffractometer. In some instances, the diffraction apparatus may be configured to record diffraction data and image data.

As used herein, the term “computer-aided diagnostic indicator” generally refers to an indicator including diagnostic information generated with the help of one or more computer processors. In some instances, the “computer-aided diagnostic indicator” may include a probability score that the subject from which an α-keratin or collagen sample was derived has a health condition, e.g., cancer or some other disease, pathological abnormalities including those resulting from environmental causes such as lead or other heavy metal poisoning, or beneficial body characteristics or conditions. In some instances, the “computer-aided diagnostic indicator” may include a diagnosis that the subject from which the α-keratin or collagen sample was derived has a health condition, e.g., cancer or some other disease, pathological abnormalities including those resulting from environmental causes such as lead or other heavy metal poisoning, or beneficial body characteristics or conditions.

As used herein, the term “subject” refers to a human or a non-human animal. A subject may be afflicted with a disease or suspected of being afflicted with or having a disease. The subject may not be suspected of being afflicted with or having the disease. The subject may be symptomatic. Alternatively, the subject may be asymptomatic. In some cases, the subject may be treated to alleviate the symptoms of the disease or cure the subject of the disease. A subject may be a patient undergoing treatment by a healthcare provider.

As used herein, the term “healthcare provider” generally refers to a medical or veterinary practitioner or support staff. The healthcare provider may be a doctor, a nurse, a dentist, a veterinarian, a technician, a student, or the like. The healthcare provider may be at least partially responsible for the healthcare of the subject.

As used herein, the term “institution” generally refers to an entity related to one or more healthcare providers. The institution may be a medical center, a doctor's office, a clinic, a hospital, a university, or the like.

As used herein, the term “cancer” generally refers to a proliferative disorder caused or characterized by a proliferation of cells which have lost susceptibility to normal growth control. Cancers of the same tissue type usually originate in the same tissue and may be divided into different subtypes based on their biological characteristics. Non-limiting examples of categories of cancer are carcinoma (epithelial cell derived), sarcoma (connective tissue or mesodermal derived), leukemia (blood-forming tissue derived) and lymphoma (lymph tissue derived). Cancer may involve any organ and tissue of the body. Specific examples of cancers that do not limit the definition of cancer may include melanoma, leukemia, astrocytoma, glioblastoma, retinoblastoma, lymphoma, glioma, Hodgkin's lymphoma, and chronic lymphocytic leukemia. Examples of organs and tissues that may be affected by various cancers include pancreas, breast, thyroid, ovary, uterus, testis, prostate, pituitary gland, adrenal gland, kidney, stomach, esophagus, rectum, small intestine, colon, liver, gall bladder, head and neck, tongue, mouth, eye and orbit, bone, joints, brain, nervous system, skin, blood, nasopharyngeal tissue, lung, larynx, urinary tract, cervix, vagina, exocrine glands, and endocrine glands. In some cases, a cancer can be multi-centric. In some cases, a cancer can be a cancer of unknown primary (CUP).

As used herein, the term “cloud” generally refers to shared or sharable storage of electronic data, e.g., a distributed network of computer servers. In some instances, the cloud may be used for archiving electronic data, sharing electronic data, and analyzing electronic data.

The present disclosure describes sample analysis and communication systems. In one example of a disclosed system, a sample may include a quantity of hair, nails, claw, hoof, skin, an internal organ, or any tissues that contains α-keratin, collagen, or similar molecular structures. In another example, a sample-analyzer subsystem includes at least one diffractometer operatively coupled to a computer database over a network, and the sample-analyzer subsystem is configured for acquisition of sample data chosen from a group including in vitro image data, in vitro diffraction pattern data, and subject data, and configured for transfer of the sample data to the computer database over the network. In still another example of the disclosed system, at least one computer processor is operatively coupled to the at least one diffractometer, and the at least one computer processor is configured to receive the subject-sample data from the at least one diffractometer, transmit the sample data to the computer database; and process the sample data using a data analytics process that provides a computer-aided objective-diagnostic indicator for a given sample. In another example, the system further includes a user interface that allows an individual subject or a healthcare provider to upload the individual subject's sample data for an in vitro sample to the computer database in exchange for processing of the sample data to receive the computer-aided diagnostic indicator for the in vitro sample or for the individual subject. In another example, the user interface is further configured to allow an individual subject or their healthcare provider to make payments or upload an individual subject's signed consent form. For example, a healthcare provider can use a user interface to upload diffraction images of a suspicious mass identified in a mammogram, along with pathology laboratory micrographs of stained breast biopsy specimens, to the computer database. In this example, the system comprising the one or more computer processors and the computer database can then process diffraction images, as well as micrograph images, using a data analytics process to generate a diagnostic indicator that is provided to the healthcare provider. In some instances, the diffraction images and the micrograph images may be retained on the computer database, where they can be used to refine the data analytics process that generates the diagnostic indicator. The user interface may be configured to allow an individual subject and/or their healthcare provider to make payments and/or upload the individual subject's signed consent form. The payments may be cash payments (e.g., the user interface displays an address to send the payments), check payments (e.g., paper or electronic check payments), card payments (e.g., credit or debit card payment processing), app-based payments (e.g., PayPal®, Venmo®), cryptocurrency payments (e.g., Bitcoin), or any combination thereof. For example, in some instances, an individual subject may pay via a health savings account debit card. The signed consent form may be signed by the individual subject, a veterinary customer, and/or the healthcare provider. The signed consent form may be related to the computer-aided diagnostic indicator. For example, the individual subject can sign and upload a consent form stating that the subject's diffraction and/or image data may be retained on the computer database. In some instances, the signed consent form may be physically signed, electronically signed, or any combination thereof.

In still another example, the system comprises at least two diffractometers, each being located in different geographic locations. In some instances, a system of the present disclosure may include 1, 2, 3, 4, 5, 6, 7, 8, 9, 10, 20, 30, 40, 50, 60, 70, 80, 90, 100, 150, 200, 250, 300, 350, 400, 450, 500, or more than 500 diffraction apparatus. In some instances, the number of diffraction apparatus in the system may range between any two of the values specified in this paragraph. For example, in some instances, the number of diffraction apparatus in the system may range from 4 to 100. Those of skill in the art will recognize that in some instances, the number of diffraction apparatus in the system may have any value within the range of values specified in this paragraph, e.g., 125 diffraction apparatus. For example, a first diffraction apparatus in a first location can send one set of image data to the one or more computer processors while a second diffraction apparatus in a second location can send one set of diffraction pattern data to the one or more computer processors. In this example, the image data and the diffraction pattern data can both be used to refine the data analytics process that generates computer-aided diagnostic indicators for individual samples and may also both be retained on the computer database.

In yet another example, the at least one diffractometer comprises a data encryption device that includes a global positioning system (GPS) positioning sensor and generates encrypted in vitro image data, in vitro diffraction pattern data, animal data, or any combination thereof, which encrypted in vitro image data, in vitro diffraction pattern data, animal data, or any combination thereof that is transferred to the computer database tracks changes in location of the one or more diffractometers. For example, the image metadata generated by a diffraction apparatus can include location information for that diffraction apparatus. In this example, a movement of the diffraction apparatus can be tracked using the image metadata transmitted by the diffraction apparatus. In another example, the GPS positioning sensor can be in constant communication with the computer database regarding the location of the diffraction apparatus. The inclusion of the GPS sensor may reduce a likelihood that the diffraction apparatus is stolen or misappropriated by untrained users. The data encryption device may be configured to encrypt the data in line with a health data privacy standard. For example, the encryption device may make the transmission and storage of the image data, diffraction pattern data, subject data, or any combination thereof compliant with the Health Insurance Portability and Accountability Act (HIPAA). The data encryption device may include a module configured to only permit communication between the diffraction apparatus and the computer database. For example, other network communications can be disabled such that the data from the diffraction apparatus can be sent only to the computer database.

In one example, the at least one diffractometer is configured to perform small angle X-ray scattering (SAXS) measurements. The SAXS measurements may include measurements of the long-range ordering of the analyzed sample. For example, the SAXS measurement can determine an order parameter, period of crystalline structure, or repeating unit size in a α-keratin or collagen sample in the range of 10 to 1,000 nanometers. The SAXS measurements may include measurements of scattering of at least about 0.01, 0.05, 0.1, 0.2, 0.3, 0.4., 0.5, 0.6, 0.7, 0.8, 0.9, 1, 1.5, 2, 2.5, 3, 3.5, 4, 4.5, 5, 5.5, 6, 6.6, 7, 7.5, 8, 8.5, 9, 9.5, 10, or more degrees. The SAXS measurements may include measurements of at most about 10, 9.5, 9, 8.5, 8, 7.5, 7, 6.5, 6, 5.5, 5, 4.5, 4, 3.5, 3, 2.5, 2, 1.5, 1, 0.9, 0.8, 0.7, 0.6, 0.5, 0.4, 0.3, 0.2, 0.1, 0.05, 0.01, or less degrees. The SAXS measurements may include measurements of a range as defined by any two of the proceeding numbers. For example, the SAXS measurements may include measurements of scattering of 0.1-10 degrees. The SAXS measurements may include measurements with respect to degrees (e.g., Θ), 2Θ, d (e.g., distance measured in Angstroms), q (e.g., 1/d), or the like, or any combination thereof.

In another example, one or more of the at least one diffractometer performs wide angle X-ray scattering (WAXS) measurements. The WAXS measurements may include measurements of the short-range ordering of the α-keratin or collagen sample. For example, the WAXS measurements can record measurements of order parameter, period of crystalline structure, or repeating unit size in the α-keratin or collagen sample below 10 nanometers. The WAXS measurements may provide structural information about non-tissue objects in the α-keratin or collagen sample. For example, a WAXS measurement of an object suspected of being a breast calcification can confirm that the object is composed of calcium oxalate and calcium phosphate. In another example, a WAXS measurement can generate information regarding a molecular structure within the α-keratin or collagen sample. The WAXS measurements may include measurements of at least about 10, 15, 20, 25, 30, 35, 40, 45, or more degrees. The WAXS measurements may include measurements of at most about 45, 40, 35, 30, 25, 20, 15, 10, or less degrees. The WAXS measurements may include measurements of a range as defined by any two of the proceeding numbers. For example, the WAXS measurements can include measurements of scattering of 10-45 degrees. The WAXS measurements may include measurements with respect to degrees (e.g., Θ), 2Θ, d (e.g., distance measured in Angstroms), q (e.g., 1/d), or the like, or any combination thereof.

In still another example, the biological samples of internal organs include a surgical sample, a resection sample, a pathology sample, a biopsy sample, or any combination thereof. In yet another example, the sample data further comprises genetic data, pathology lab image data, subject data, or any combination thereof.

In one example, the computer database resides on a central server. In another example, the computer database resides in the cloud (e.g., may be a cloud-based computer server comprising a distributed network of remote computer servers). In some instances, the computer database may reside on a local server. In some instances, data may be transferred or exchanged between a local computer database and a remote or central computer database. The computer database may reside on a privacy law compliant server (e.g., a HIPAA complaint server).

In still another example, the sample data (the image data, diffraction pattern data, subject data, or any combination thereof) transferred to the computer database are depersonalized prior to the transfer. The depersonalization may include removal of personally identifiable information (e.g., name, patient number, social security number, address, etc.). For example, identifying information can be removed from image metadata and/or subject data before the image metadata and/or subject data are transferred to the computer database. The depersonalization of the image data, diffraction pattern data, subject data, or any combination thereof may aid in making the computer database compliant with privacy laws. In yet another example, a key for mapping the depersonalized sample data stored in the computer database to an individual subject is stored in a local institutional database or in the individual subject's personal files. For example, a key can be generated that relates a subject to their depersonalized data for later reference or reunification. The local institutional database may be a database operated by the institution where the subject went to obtain the image data, diffraction pattern data, subject data, or any combination thereof. For example, a hospital can have a database comprising keys to link the identities of hospital patients to their depersonalized data. In another example, the key can be kept in the patient's personal medical files.

In one example, the data analytics process includes a statistical analysis of diffraction pattern data or a function thereof including but not limited to regression analysis, spline approximation, or Fourier series approximation. In some instances, the data analytics process may include a statistical analysis of image data, diffraction pattern data, subject data, a function of any of the proceeding, or any combination thereof. In another example, the statistical analysis comprises determination of a pair-wise distance distribution function, determination of a Patterson function, a calculation of a Porod invariant, a cluster analysis, a factor analysis, a dispersion analysis, determination of one or more molecular structural periodicities, or any combination thereof. In still another example, the statistical analysis comprises a determination of a structural periodicity of α-keratin and/or collagen. In yet another example, the statistical analysis comprises a determination of a structural periodicity of one or more lipids. In one example, the statistical analysis comprises a determination of a structural periodicity of the α-keratin and/or collagen samples and/or biological samples of internal organs of a human or a non-human animal. For example, a diffraction pattern can provide information regarding the structural periodicity, and thus the relative degree of ordering, of the collagen and α-keratin within the spot size of the diffractometer. In another example, the ordering of lipid layers can be determined by diffraction, which can give information about the stiffness of the lipid layers and the chemical composition of the layers (e.g., the amount of cholesterol or other stiffening agents) on a local level. In some cases, the structural periodicity of α-keratin and collagen samples and biological samples of animal internal organs can be used to determine the probability of a health condition such as cancer or other disease in a patient or any pathological abnormalities including cases caused by environmental, i.e., heavy metal poisoning such as lead poisoning.

In another example, the data analytics process includes a machine learning process selected from the list including a supervised learning process, an unsupervised learning process, a semi-supervised learning process, a reinforcement learning process, a deep learning process, or any combination thereof. The one or more machine learning process may be configured to operate upon image data, diffraction pattern data, subject data, or any combination thereof. In yet another example, in the machine learning process uses a deep learning process. In one example, the deep learning process is a convolutional neural network, a recurrent neural network, or a recurrent convolutional neural network.

Statistical analysis and/or machine learning processes implemented on a local computer, or a remote server may perform data analytics. For example, a machine learning process can be configured to pre-process raw image data, diffraction pattern data, and/or subject data to remove noise or other artifacts. A different machine learning can be trained to identify features within the image data, diffraction pattern data, and/or subject data. Such a machine learning processes can cluster data points for use as an identification process. Other machine learning techniques can be configured to provide a computer-aided diagnostic indicator.

The machine learning may include a supervised, semi-supervised, or unsupervised machine learning techniques. Supervised machine learning, for example, may be trained using labeled training data sets, e.g., data sets that include training inputs with known outputs. The training inputs can be provided to an untrained or partially trained version of the machine learning system to generate a predicted output. The predicted output can be compared to the known output in an iterative process, and if there is a difference, the parameters of the machine learning system can be updated. A semi-supervised machine learning process is trained using a large set of unlabeled training data, e.g., unlabeled training inputs, and a small number of labeled training inputs. An unsupervised machine learning process, e.g., a clustering process, may find previously unknown patterns in data sets comprising data with no pre-existing labels.

One non-limiting example of a machine learning process that can be used to perform some of the functions described above, e.g., processing of diffraction data, image data, and/or generating computer-aided diagnostic indicators, is a neural network. Neural networks employ multiple layers of operations to predict one or more outputs, e.g., a likelihood that a subject has cancer, from one or more inputs, e.g., image data, diffraction pattern data, subject data, processed data derived from image data, diffraction pattern data, and/or subject data, or any combination thereof. Neural networks can include one or more hidden layers situated between an input layer and an output layer. The output of each layer can be used as input to another layer, e.g., the next hidden layer or the output layer. Each layer of a neural network can specify one or more transformation operations to be performed on the data input to the layer. Such transformation operations may be referred to as “neurons.” The output of a particular neuron may be, for example, a weighted sum of the inputs to the neuron that is optionally adjusted with a bias and/or multiplied by an activation function, e.g., a rectified linear unit (ReLU) or a sigmoid function.

Training a neural network can involve providing inputs to the untrained neural network to generate predicted outputs, comparing the predicted outputs to expected outputs, and updating the weights and biases in an iterative manner to account for the difference between the predicted outputs and the expected outputs. For example, a cost function can be used to calculate a difference between the predicted outputs and the expected outputs. By computing the derivative of the cost function with respect to the weights and biases of the network, the weights and biases can be iteratively adjusted over multiple cycles to minimize the cost function. Training may be complete when the predicted outputs satisfy a convergence condition, such as obtaining a small magnitude of calculated cost.

Convolutional neural networks (CNNs) and recurrent neural networks can be used to classify or make predictions from image data, diffraction pattern data, subject data, or any combination thereof. CNNs are neural networks in which neurons in some layers, called convolutional layers, receive data from only small portions of a data set. These small portions may be referred to as the neurons' receptive fields. Each neuron in such a convolutional layer may have the same weights. In this way, the convolutional layer can detect features, e.g., cancerous growths, in any portion of the input image data, diffraction data, or a combination thereof.

Recurrent neural networks (RNNs), meanwhile, are neural networks with cyclical connections that can encode dependencies in time-series data, e.g., longitudinal study images of one or more subjects. An RNN may include an input layer that is configured to receive a sequence of time-series inputs, e.g., image data, diffraction pattern data, subject data, or any combination thereof collected over a period of time. An RNN may also include one or more hidden recurrent layers that maintain a state. At each time step, each hidden recurrent layer can compute an output and a next state for the layer. The next state can depend on the previous state and the current input. The state can be maintained across time steps and can capture dependencies in the input sequence. Such an RNN can be used to determine time-series features or evolutions of features within the subject data.

One example of an RNN is a long short-term memory network (LSTM), which can be made of LSTM units. An LSTM unit can be made of a cell, an input gate, an output gate, and a forget gate. The cell can be responsible for keeping track of the dependencies between the elements in the input sequence. The input gate can control the extent to which a new value flows into the cell, the forget gate can control the extent to which a value remains in the cell, and the output gate can control the extent to which the value in the cell is used to compute the output activation of the LSTM unit. The activation function of the LSTM gate may be, for example, the logistic function.

Other examples of machine learning processes that can be used to process image data, diffraction pattern data, subject data, or any combination thereof are regression, decision trees, support vector machines, Bayesian networks, clustering processes, reinforcement learning processes, and the like.

The clustering processes may be, for example, a hierarchical clustering process. A hierarchical clustering process can be a clustering process that clusters objects based on their proximity to other objects. For example, a hierarchical clustering process can cluster image data, diffraction pattern data, subject data, or any combination thereof. The clustering process can alternatively be a centroid-based clustering process, e.g., a k-means clustering. A k-means clustering process can partition n observations into k clusters, where each observation belongs to the cluster with the nearest mean. The mean can serve as a prototype for the cluster. In the context of image data, diffraction pattern data, subject data, or any combination thereof, k-means clustering can generate distinct groups of data that are correlated with each other. Thereafter, each group of data can be associated with, e.g., a particular probability or diagnosis of a health condition, e.g., cancer or other disease, based on knowledge about that subsystem, e.g., knowledge about previous diagnoses and data. The clustering can alternatively be a distribution-based clustering, e.g., a Gaussian mixture model or expectation maximization process. Examples of other clustering techniques may use cosine similarity, topological data analysis, and hierarchical density-based clustering of applications with noise (HDB-SCAN).

In another example, the machine learning process is trained using a training dataset comprising pathology lab image data, diffraction pattern data, subject data, or any combination thereof from one or more control samples. In still another example, the training dataset is updated as new sample data are uploaded to the computer database. In yet another example, the sample data further comprises subject data comprising an individual subject's species, age, sex, weight, body condition score (BCS), ancestry data, genetic data, behavioral data, or any combination thereof, wherein the sample is from the individual subject. The training dataset may be stored in the computer database for a specific pathology and/or physiological norm group. The training dataset may be obtained using the one or more diffraction apparatus. The training dataset may include micrograph images of stained tissue specimens. The training dataset may include information regarding a confirmation of a diagnosis for given set of data. For example, data including multiple images and diffraction patterns of a tissue suspected of being cancerous can also include a histological confirmation of the presence of the cancer in the tissue. In another example, a set of diffraction images can be accompanied by data regarding the longevity of the subject from whom the diffraction images were taken.

The computer database for the specific pathology and/or physiological norm group may be a remote computer database (e.g., a cloud-based database) or a local database (e.g., a computer system local to a diffraction apparatus). For example, the training dataset for breast cancer diagnostic indicators can be stored on a computer database with other breast cancer diagnostic data. The training dataset may be updated as new image data, diffraction pattern data, subject data, or any combination thereof is uploaded to the computer database. The updating may be an addition of the new data, a removal of the old data, or a combination thereof. For example, new image data can be added to the training dataset as it is taken to improve the quality of the training dataset. In another example, poor quality data may be removed from the training dataset when higher quality new data is added. The statistical analysis and/or machine learning process (e.g., the data analytics) may be updated when the computer database or training dataset residing thereon is updated. For example, a machine learning process can be retrained using the new training dataset to improve the efficacy of the machine learning process in generating a computer-aided diagnostic indicator. The statistical analysis and/or machine learning process may be continuously, periodically, or randomly updated and refined as the training dataset is updated. In this example, the revised statistical analysis and/or machine learning process may be more accurate, specific, and/or sensitive in providing a probability or diagnosis than a previous version derived from a previous training dataset was.

In one of the examples, the computer-aided diagnostic indicator for the in vitro sample includes an indicator of a likelihood that the sample indicates a presence or absence of cancer or another disease or a pathological abnormality of subject (patient). For example, the computer-aided diagnostic indicator for an individual subject may include an indicator of a likelihood that the individual subject has breast cancer. A computer-aided diagnostic indicator can include a banded risk assessment for the individual subject (e.g., high risk, medium risk, low risk). The computer-aided diagnostic indicator may be displayed on a user interface of a device (e.g., a user interface on a computer screen or on a tablet). The computer-aided diagnostic indicator may be a report. The report may be a printed report. The report may include information in addition to a computer-aided diagnostic indicator. For example, the report may include a likelihood of the subject having a cancer, as well as the indicators that contributed to the generation of the report and a suggestion of possible next steps for the subject to take. The indicator may be a percentage (e.g., a percentage likelihood that the subject has the cancer), a risk band (e.g., high risk, medium risk, low risk), a comparison of factors (e.g., a list of factor indication a presence and a list of factors indicating an absence), or the like, or any combination thereof. The indicator of the likelihood that the individual subject has cancer may be an indicator of the likelihood that the individual subject has breast cancer.

In some instances, the computer-aided diagnostic indicator for the individual subject may include a diagnosis that the individual subject has a cancer or other disease. The computer-aided diagnostic indicator for the individual subject may include a diagnosis that the individual subject has breast cancer. The computer-aided diagnostic indicator may be generated at least in part using a statistical analysis and/or a machine learning process. The computer-aided diagnostic indicator may be generated at least in part using input from a healthcare provider. For example, the healthcare provider can be presented with a list of indicators and risk bands, and the healthcare provider can make a final determination as to the diagnosis of the subject. In some instances, the computer-aided diagnostic indicator may have an accuracy, selectivity, and/or specificity of at least about 10%, 20%, 30%, 40%, 50%, 60%, 70%, 80%, 90%, 95%, 98%, 99%, 99.9%, or better. In some instances, the computer-aided diagnostic indicator may have an accuracy, selectivity, and/or specificity of at most about 99.9%, 99%, 98%, 95%, 90%, 80%, 70%, 60%, 50%, 40%, 30%, 20%, 10%, or less. Any of the lower and upper values described in this paragraph may be combined to form a range included within the present disclosure, for example, in some instances the computer-aided diagnostic indicator may have an accuracy, selectivity, and/or specificity that ranges from about 80% to about 99%. Those of skill in the art will recognize that, in some instances, the computer-aided diagnostic indicator may have an accuracy, selectivity, and/or specificity that has any value within this range, e.g., about 98.6%.

In another example, the diagnosed disease may be, but is not limited to, breast cancer, brain cancer, bone cancer, lung cancer, cervical cancer, bladder cancer, head and neck cancer, kidney cancer, intestinal cancer, liver cancer, ovarian cancer, pancreatic cancer, prostate cancer, skin cancer, throat cancer, oral cancer, vaginal cancer, or any combination thereof. In still another example, the pathology lab image data comprises micrographs of stained in vitro tissue specimens. In yet another example, the system is used to monitor the efficacy of a cancer therapeutic treatment.

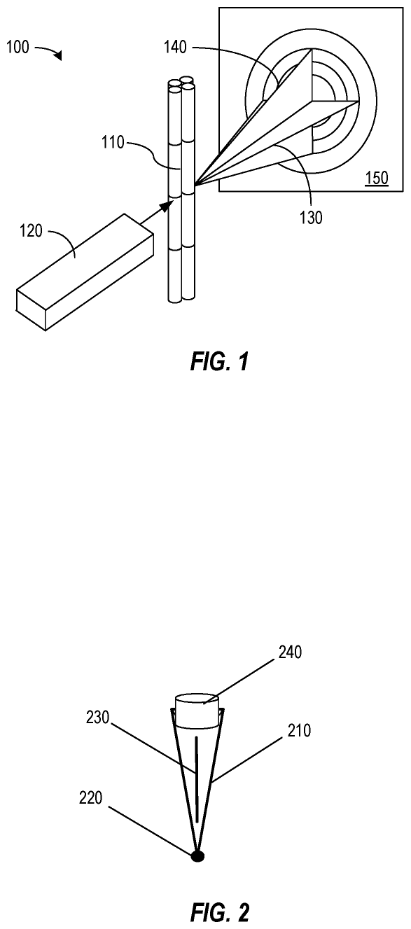

The α-keratin and collagen contained in animal hair, nails, skin and biological samples of animal internal organs contain pseudo-crystalline rod-like structures which can be studied by fiber diffraction. FIG. 1 schematically shows a diffractometer 100 having well oriented fibers 110 placed in an incoming collimated X-ray beam from a source 120, so that the parallel fibers 110 are at right angles to the beam. FIG. 1 also illustrates scattered rays in equatorial and meridional directions or planes 130 and 140. A sensor 150 can measure a 2-D scattering pattern of the X-rays that are diffracted at specific angles, e.g., angles less than about 10 degrees and produce diffraction pattern data that can be recorded and analyzed. Diffraction study of such fibers 110 may be achieved in minutes using laboratory-based X-ray tubes with rotating anodes combined with multilayer optics as X-ray source 120. Such equipment has been used to examine changes from normal tissue in pathological tissue specific to a disease of that tissue, and fiber diffraction techniques have been used in the study of muscle, collagen, and keratin.

A sample, e.g., a hair sample, for fiber diffraction measurements can be prepared as follows. (See FIG. 2.) For this purpose, a glass capillary tube with a diameter of about 2 to 3 mm may be used. One end of the tube is heated to the state of melting glass and then the tube is stretched to form a funnel 210. Glass funnel 210 may, for example, have an upper diameter of 2 to 3 mm and a lower diameter of approximately 1 mm. The lower end of funnel 210 may be closed up by a molten glass droplet 220. The result is a container 210 with a length of about 2 cm. Hair segments 230 approximately 1 cm in length may be placed in container 210. The container 210 is then sealed with a stopper 240 to maintain a constant humidity inside container 210. In such a container 210, hair segments 230 do not experience deformation stresses, which is very important when conducting X-ray diffraction studies. Twisted or curly hair may be wetted and smoothed before preparing of hair segments 230 for diffraction measurements.

The molecular structures of hair of a subject with cancer are likely related with changes in tissues remote from the affected area that could be associated with the malignancy. Different malignancies have been found to have specific ring patterns that are superimposed on the normal hard α-keratin pattern. Because the keratin pattern itself remains unaltered for hair from patients with cancer, the additional rings, radii specific to the cancer type, indicate that the randomly arranged extra material in the hair, which gives rise to these extra rings, is not associated in any way with the helical sections of the α-keratin. Also, diseases such as insulin dependent diabetes mellitus can change the “normal” diffraction pattern of hair where the intensity distribution of the meridional arcs is altered, and the radius of the intermediate filaments is increased indicating that material is bound to the helical section of α-keratin.

Nail samples from human subjects may be prepared as described in the above-cited article of Veronica J. James. Claw or hoof samples from animal subject may be similarly prepared. Nails, claws, and hooves generally contain of rigid and durable dense keratinized plates. The clippings from such samples may be polished on both sides, using alternately the rough, soft and silky sides of the polishing block. For diffraction measurements, small flat sections of samples, approximately 1 mm×3.2 mm, are cut with the shorter length in the direction of the keratin fibers. These “crystallites” may be anchored to the ends of quartz microtubules or pins that are then attached to a plate, specially grooved to accommodate the microtubules or pins. Attachment to at both ends of such a sample may be unnecessary because the samples are rigid. The “crystallites” may be attached to goniometers to further assist alignment. This size of human nail sample eliminates the inherent curvature and as a consequence eliminates disorder resulting from the curvature. The sample holders are positioned so that the sample to be irradiated protruded beyond the edge of the plate in the direction of growth of the keratin fibrils thus giving rise to the meridional (vertical) pattern for keratin. The arcs in the meridional pattern result from the repeat distances within the helical sections of α-keratin fibrils giving rise to the two infinite lattices with repeat spacings of 46.7 nm and 62.6 nm. The equatorial (horizontal) pattern of spots in a resulting diffraction pattern from measurement of the sample arises from the transverse cylindrical packing of the intermediate filaments of the keratin.

Preparation of skin samples also described in the above-cited article of Veronica J. James. Skin samples may be obtained from routine surgery, necropsies or from punch biopsies. Unless the skin sample is removed during routine surgery for the carcinoma, skin samples may be taken from the underarm of patients because of the relatively low level of ultraviolet exposure/damage at this site. Participants of a control groups may be selected during routine medical examinations in such a way that they do not have the studied diseases and other skin complaints. No prior treatment is required before harvesting of the sample as the surface layers of skin are removed from the sample and not used in diffraction measurements. The diffraction measurement may only use the dermal layer.

Immediately after excision, the skin samples may be placed in physiological saline and stored at 220° C. until required. Tests have shown that no deterioration occurs in samples stored in this way for up to 12 months. Before mounting in the cells, which have been specifically designed to maintain 100% humidity throughout the experiment, the skin samples, approximately 1 mm by 3.5 mm in size, are gently scraped to remove the epithelial and epidermal layers and so to expose the dermal layer. These cells also allow for the skin samples to be stretched slightly, removing the natural crimp, using the sutures surgically applied to the 1 mm ends. The 2D collagen sample is thereby aligned preferentially in the direction of the applied stretch thus giving rise to the arcs in the meridional (vertical) pattern. The angular spread of the arcs is determined by the degree of alignment. If perfectly aligned, spots, not arcs, would be seen. The meridional lattice spacing for wet skin from a control group may be about 65.2 nm. The equatorial (horizontal) pattern reflects the cylindrical packing arrangement of the collagen.

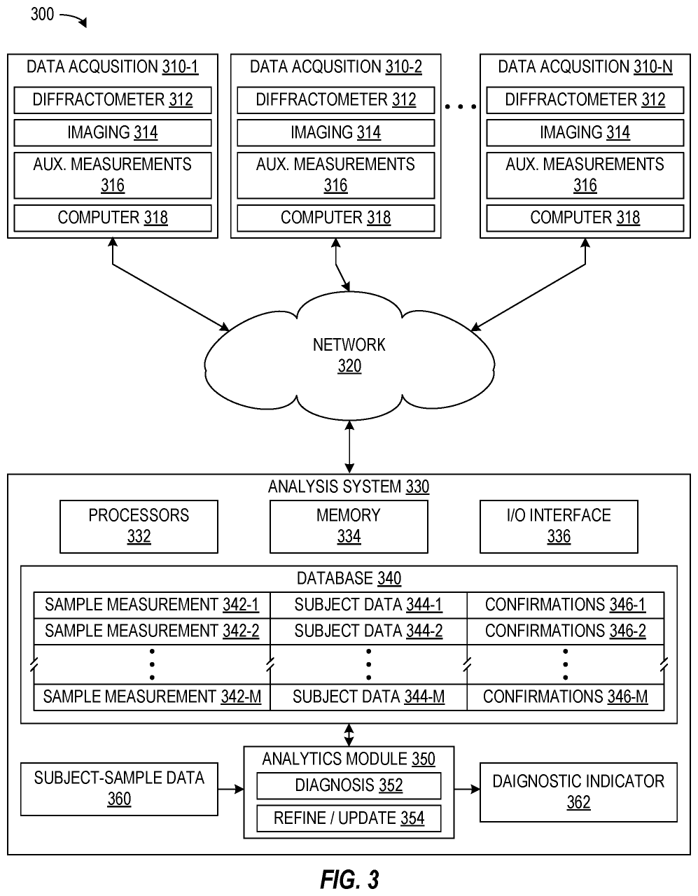

FIG. 3 is a block diagram showing a diagnostic system 300 in accordance with an example of the present disclosure. Diagnostic system 300 includes multiple data acquisition systems 310-1 to 310-N, generically referred to herein as data acquisition systems 310. Data acquisition systems 310-1 to 310-N may reside at different geographical locations. For example, each system 310 may be at a different medical or veterinarian office or hospital or different dedicated medical of veterinarian test facilities. Each data acquisition system 310 includes one or more diffractometer 312 suitable for measuring a sample to obtain diffraction data for the sample as described above. Each data acquisition system 310 may further include additional sample measurement systems such as an imaging system 314, e.g., a microscope or a camera, capable of capturing image data from a sample to be subjected to diffraction measurements or from tissue related to the sample. For example, an imaging system 314 may include a microscope that captures images showing cell structure in a sample before diffraction measurements. Data acquisition systems 310 may include further measurement systems 316 to measure other aspects of the sample or subject, for example, genetic or chromosome characteristics.

Each data acquisition system 310 further includes a computer system 318 that is connected to the local diffractometer 312 and other local measurement systems 314 and 316. Computer system 318 may include hardware specialized for diffraction and other sample measurements or may be a general-purpose computer executing programs that implement processes described herein. Computer system 318 may, for example, be used for data entry or to otherwise obtain personal or depersonalized subject data for a subject providing a sample being analyzed. Subject data may, for example, indicate an individual human or animal subject's species, age, sex, weight, body condition score (BCS), ancestry data, genetic data, behavioral data, or any combination thereof. Subject data may also include personal data that identifies the subject or identifies a person associated with the subject, e.g., the parent of a child or the owner or caretaker of an animal. Computer system 318 may further depersonalize the subject data and format, encrypt, or compress and transmit the subject data and measurement data from measurements of the sample to an analysis system 330. In the illustrated example, a network 320 interconnects data acquisition systems 310-1 to 310-N and analysis system 330. Network 320 may be a conventional computing network that may include one or more private network, e.g., local wired or wireless networks at the sites were data acquisition systems 310 or analysis system 330 reside, and one or more public network, e.g., the Internet.

Analysis system 330 may be a global diagnostics system that provides diagnostic processing or services for users located anywhere in the world. Analysis system 330 may include a computer or server system including one or more computers or servers with conventional components such as processors 332, memory 334, and I/O interfaces 336 used to execute programs and implement processes and modules discloses herein. Analysis system 330 may be a local computer database (e.g., a local computing cluster housed in the same facility as where the data was acquired) or a remote computer database (e.g., a cloud computing database).

Analysis system 330 provides a database 340 containing sample and subject data from a large number of samples and subjects. Computer database 340 may particularly be cond to store sample measurements 342, e.g., image data, diffraction pattern data and or subject data 344 and 346 or any combination thereof. Database 340 may be encrypted for protection of personal information of subjects or other users. In particular, database 340 may be configured for compliance with health data privacy laws and regulations (e.g., HIPAA). Database 340 may be a distributed computer database (e.g., a cloud-based database housed at multiple locations).

Analysis system 330 may accept data for database 340 from one or more diffractometers 312 via one or more associated computer systems 318, and analysis system 330 and/or a computer system 318 may be configured to pre-process, process, and/or post-process sample and subject data as described herein. In different configurations, analysis system 330 includes one or more computer processors 332 coupled to receive data from one or more diffraction apparatus or diffractometer 312 via network 320, e.g., one or more of a local network, the Internet, and a virtual private network. The one or more diffraction apparatus 312 may include at least about 1, 5, 10, 25, 50, 75, 100, 250, 500, 750, 1,000, 2,500, 5,000, 10,000, 50,000, 100,000, or more diffraction apparatus. The one or more diffractometers 512 may include up to about 100,000, 50,000, 10,000, 5,000, 2,500, 1,000, 750, 500, 250, 100, 75, 50, 25, 10, 5, or fewer diffraction apparatus. One or more of diffractometers 312 may be the same type of diffraction apparatus (e.g., a same model), or one or more of diffractometers may be different (e.g., one or more different models of diffraction apparatus).

Analysis system 330 includes an analytics module 350 that receives subject-sample data 360 from a data acquisition system 310, use subject-sample data 360 and data derived from database 340 in a diagnosis process 352 to generate an objective diagnostic indicator 362 for the subject associated with the subject-sample data 360, and further uses subject-sample data 360 in a refinement or update process 354. Diagnosis process 352 may operate to categorize incoming subject-sample data 360 into a class that was found in database 340 a probability of confirmation 346 of a condition and generates diagnostic indicator 362 based on the confirmations 346 for the class. Refinement/update process 360 may periodically update database 340 and refine statistical and/or machine learning based data analytics processes that define diagnosis process 350. For example, diagnosis process 350 may be updated every month, every week, every day, or every hour based on changes in database 340. In some instances, analytics module 350 and database 350 may be configured to continually refine a statistical and/or machine learning based data analytics process that defines diagnosis 352. For example, each time analysis system 350 receives new subject-sample data 360 including measurement data from a diffractometer 312, refinement/update process 354 can add the new data to computer database 240 and process database 340 to update the data analytics process used in diagnosis process 352. The refinement may employ a data analytics process and/or machine learning process as described herein.

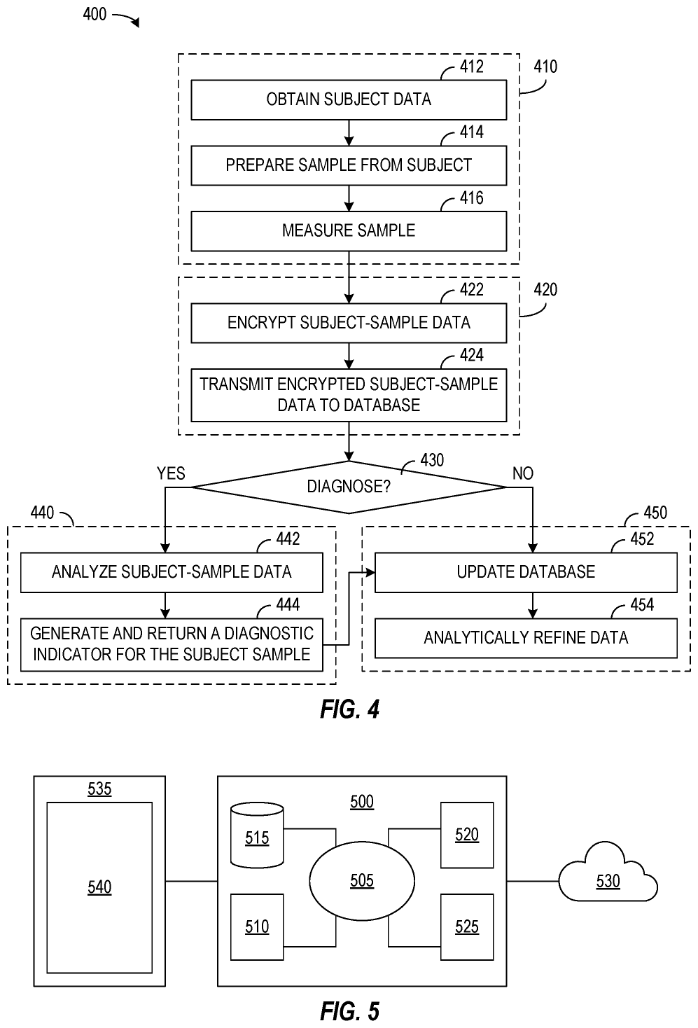

FIG. 4 shows workflow a data collection and analysis process 400. In a data acquisition operation 410, process 400 may acquire diffraction data, image data, subject data, or any combination thereof for in vitro samples derived from an individual subject, e.g., from a human or non-human animal. In an operation 412, subject data such as species, age, sex, weight, body condition score (BCS), ancestry data, genetic data, behavioral data, or any combination thereof may be obtained from available records, the subject, or a parent or caretaker of the subject, and the subject data may be entered into a data acquisition system. An operation 414 may then prepare a sample from the subject. For example, a sample such as a clipping or shaving of nail from a human subject a claw or hoof of animal subject or a clipping of hair or a tissue sample from the subject may be taken and mounted in a sample holder for diffraction measurements. A measurement process 426 measures the sample to at least produce diffraction data. The diffraction data may represent relative or absolute intensities at 2-D angular coordinates or in equatorial and meridional planes or indicate 2-D angular coordinates or equatorial and meridional angles identifying the locations of local maxima in a diffraction pattern produced using the sample. More generally, measuring the sample to acquire data may use a microscope, a diffraction-based instrument, or a combination of other tissue characterization instruments and diffraction apparatus.

An operation 420 of process 400 may transfer subject-sample data to an analysis system. For example, a data acquisition system, in an operation 422, may encrypt some or all of the subject-sample data before transmitting, in an operation 424, the encrypted data to a computer database. The encrypted data may include image data, diffraction pattern data, subject data, or any combination thereof for one or more individual subjects. For example, the encrypted transferred data can include all data taken from a radiology clinic in a day. In another example, the encrypted data can be data for in vitro samples from an individual subject served by a radiology clinic. The encrypted data may be encrypted using an asymmetric key encryption, a symmetric key encryption, or the like. The encrypted data may be encrypted by a computing device local to where the data was generated (e.g., a computer 318 operatively coupled to a diffraction apparatus 312 in a data acquisition system 300 of FIG. 3). The encrypted data may be stored locally before being transferred to the analysis system or database. The encrypted data may be streamed (e.g., transferred in real-time or substantially real-time) to the analysis system or computer database. The encrypted data may be uncompressed data or uncompressed data.

The transmitted subject-sample data may be data for which a diagnosis is sought or may data that is intended solely for improvement of the analysis system and database. A decision process 430 identifies whether a diagnosis sought.

A diagnosis process 440 of process 400 may include processing data for the individual subject using a data analytics process. The processing may be performed on one or more computer processors as described elsewhere herein. The processing may be encoded on a non-transitory computer readable medium. The data analytics process may be a statistical analysis process and/or a machine learning process. The data analytics process may be a convolutional neural network as described elsewhere herein. The data analytics process may perform pre-processing, processing, and/or post-processing of diffraction data, image data, subject data, or any combination thereof. The pre-processing 442 may include denoising (e.g., removing noise or errors from the data), normalizing (e.g., standardizing data properties such as size, black level, maximum intensity, etc.), segmentation (e.g., dividing the data into sections comprising different features), masking (e.g., applying one or more masks to the data), enhancing edges and/or features, or the like, or any combination thereof. The processing may include determining a presence or absence of a feature in the data (e.g., determining a presence of a feature indicative of a health condition such as a cancer), determining a severity of a feature in the data (e.g., determining the progression or stage of a cancer), clustering data (e.g., clustering images based on the presence or absence of a feature), predicting a presence or absence of a feature in new data (e.g., using previously acquired images to generate a prediction of a presence of a feature in a new set of data), or the like, or any combination thereof. The post-processing 444 may include formatting (e.g., formatting data for presentation to a subject or a healthcare worker), denoising, normalizing, masking, enhancing properties (e.g., contrast, edges), or the like, or any combination thereof.

An operation 444 of process 900 may include generating a diagnostic indicator for the individual sample. The diagnostic indicator may be a computer-aided diagnostic indicator. The computer aided diagnostic indicator may be a report intended for a machine, a human readable, or both to read. For example, the computer aided diagnostic indicator can be a report displayed on a user interface of a device. The diagnostic indicator may include information about a likelihood of a presence of a health condition in the subject (e.g., a cancer), a severity of a presence of a health condition (e.g., a prognosis based on the indicated severity or stage of the cancer), one or more suggested treatments (e.g., a suggestion of a mastectomy for a severe breast cancer), additional information (e.g., locations of resources to help the subject understand the diagnostic indicator), subject data (e.g., the name of the subject the indicator is for), or the like, or any combination thereof. The diagnostic indicator may be generated on the same computer system that ran the data analytics process 442 or 444. The diagnostic indicator may be held until the healthcare provider provides an input. The input may be a payment (e.g., a payment from the subject, a parent or caretaker of the subject, or a payment from the subject's insurance), an agreement for the sample data for an individual subject to be used for training and/or validating future data analytics process, or the like, or any combination thereof. For example, the subject or a parent or caretaker for the subject may be informed that the diagnostic indicator is ready and may sign a waiver allowing use of the subject's data.

Another operation 450 of process 400 may include updating the computer database with the image data, diffraction data, subject data, or any combination thereof generated for one or more subjects using one or more diffraction apparatuses. The updating may make additional data available to train a new data analytics process or update an existing data analytics process. The computer database may be updated with a confirmation of an indication made in a diagnostic indicator. For example, the database can be updated with information regarding the surgical confirmation of health condition (e.g., cancer) in a subject for whom the diagnostic indicator indicated a likelihood of the health condition (e.g., cancer). This updating may provide a confirmation of positive or negative results that can improve the accuracy of future diagnostic indicators. The data may be agglomerated for multiple subjects to generate a general classifier. For example, a database of stained tissue micrographs and diffraction patterns for breast biopsy samples can be used to generate a classifier for breast tissue. In another example, a database of brain tissue images and diffraction patterns can be used to generate a classifier for brain tissues.

Another operation 454 of process 400 may include refining the data analytics processes 440. The refining may include generating a new data analytics process or new rules for data analytics processes. The refining may include an updating of weights or other components within the data analytics process. For example, the neural weights of a neural network can be updated based on the additional data for in vitro samples derived from the subject-sample data or confirmations newly added to the database. The refining of the data analytics process may improve the sensitivity, specificity, accuracy, or any combination thereof of the data analytics process. The refined data analytics process may be used to process the data for tissue samples from another subject (e.g., used as the data analytics process of operation 440).

Another application of the disclosed methods and systems is monitoring of therapeutic efficiency for treatment of the health condition, e.g., the efficiency of cancer or other diseases treatment. Tissue samples collected from a patient after a period of receiving a therapeutic treatment are analyzed and evaluated for changes in sample data characteristics and clustering. As a result, the data analytics process may, for example, plot patient sample data points in an n-dimensional space defined by two or more treatment parameters that describe the clustering of the sample data, and the distance or changes in distance between different clusters is calculated as a function of time. In some instances, for example, the proximity of a new data point to the previous data point(s), or the trajectory of certain data clusters (or the gradient of the trajectory) may be used as an indicator for the therapy's effectiveness and can be interpreted by physician in terms of therapeutic efficiency. Comparing the results of follow up assessments for multiple patients' samples may provide indications of the efficiency of certain drugs and treatments in particular groups of patients.

Each of modules disclosed herein may include, for example, hardware devices including electronic circuitry for implementing the functionality described herein. In addition or as an alternative, each module may be partly or fully implemented by a processor executing instructions encoded on a machine-readable storage medium.

The present disclosure also provides computer systems that are programmed to implement methods of the disclosure. FIG. 5 shows a computer system 500 that is programmed or otherwise configured to implement methods described elsewhere herein (e.g., obtaining data from one or more diffraction apparatus, processing the data, etc.). The computer system 500 can regulate various aspects of the present disclosure, such as, for example, the processing of diffraction pattern data, subject data, or any combination thereof. The computer system 500 can be an electronic device of a user or a computer system that is remotely located with respect to the electronic device. The electronic device can be a mobile electronic device. The computer system 500 may be a post-classical computer system (e.g., a quantum computing system).

The computer system 500 includes a central processing unit (CPU, also “processor” and “computer processor” herein) 505, which can be a single core or multi core processor, or multiple processors for parallel processing. The computer system 500 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520, and peripheral devices 525 are in communication with the CPU 505 through a communication bus of a motherboard. The storage unit 515 can be a data storage unit (or data repository) for storing data. The computer system 500 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 500, can implement a peer-to-peer network, which may enable devices coupled to the computer system 1301 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 500 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 500 in some cases can include one or more additional data storage units that are external to the computer system 500, such as located on a remote server that is in communication with the computer system 500 through an intranet or the Internet.

The computer system 500 can communicate with one or more remote computer systems through the network 530. For instance, the computer system 500 can communicate with a remote computer system of a user (e.g., a cloud server). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 500 via the network 530.

Methods as described herein can be implemented by way of machine (e.g., computer processor) executable code stored on an electronic storage location of the computer system 500, such as, for example, on the memory 510 or electronic storage unit 515. The machine executable or machine-readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 500, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 500 can include or be in communication with an electronic display 535 that implements at least part of a user interface (UI) 540 for providing, for example, an interface for a healthcare or an individual subject to upload image data, diffraction pattern data, subject data, or any combination thereof to a computer database. Examples of UI's include, without limitation, a graphical user interface (GUI) and web-based user interface.

While specific systems and processes have been shown and described herein, it will be obvious to those skilled in the art that such systems and processes are provided by way of example only. It is not intended that the invention be limited by the specific examples provided within the specification. The descriptions and illustrations herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the systems and processes described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A diagnostic system comprising:
a data acquisition system that measures a sample from a subject and produces subject-sample data including measurements of the sample; and
a server system connected to receive the subject-sample data from the data acquisition system, the computer system implementing an analysis module producing an objective-diagnostic indicator by analyzing of the subject-sample data and data derived a database containing subject-sample data for samples from a plurality of prior subjects.

2. The system of claim 1, wherein the sample contains at least one of α-keratin and collagen from hair, nails, claws, hooves, skin, tissue, and biological samples of internal organs of the subject.

3. The system of claim 1, wherein the subject-sample data comprises one or more of diffraction pattern data, in vitro image data, subject data, genetic data, and pathology lab image data.

4. The system of claim 1, wherein the data acquisition system comprises a diffractometer, the measurements of the sample including diffraction pattern data measured using the sample.

5. The system of claim 4, wherein the data acquisition system further comprises a computer system configured to receive the measurements of the sample from the diffractometer, transmit the subject-sample data to the server database, the server system processing the subject-sample data using a data analytics process that provides the objective-diagnostic indicator based on the sample.

6. The system of claim 5, wherein the computer system provides a user interface that allows a user to transmit the subject-sample data to the server system.

7. The system of claim 6, wherein the user interface is further configured to allow the user to upload a signed consent form or make payments to the server system.

8. The system of claim 4, wherein the data acquisition system further comprises a data encryption device encrypting the subject-sample data before transmission to the server system.

9. The system of claim 4, wherein the data acquisition system further comprises a data encryption device that includes a global positioning system (GPS) device, the subject-sample data including GPS data identifying a location where the sample was measured.

10. The system of claim 4, wherein the diffractometer is configured to perform small angle X-ray scattering (SAXS) measurements.

11. The system of any one of claim 4 wherein the diffractometer is configured to perform wide angle X-ray scattering (WAXS) measurements.

12. The system of claim 1, further comprising one or more additional data acquisition systems configured to measure samples and transmit subject-sample data to the server system, the additional data acquisition systems being at different geographic locations from the data acquisition system.

13. The system of claim 1, wherein the sample comprises one or more of a surgical sample, a resection sample, a pathology sample, and a biopsy sample.

14. The system of claim 1, wherein the database resides in the cloud.

15. The system of claim 1, wherein the subject-sample data is depersonalized prior receipt by the server system.

16. The system of claim 15, wherein a key for mapping the depersonalized subject sample data in the database to the subject is stored in one of a local institutional database or in personal files of a person responsible for the subject.

17. The system of claim 1, wherein the analysis module performs a statistical analysis of diffraction pattern data or a function of diffraction pattern data.

18. The system of claim 17, wherein the statistical analysis comprises determination of a pair-wise distance distribution function, determination of a Patterson function, a calculation of a Porod invariant, a cluster analysis, a factor analysis, a dispersion analysis, determination of one or more molecular structural periodicities, or any combination thereof.

19. The system of claim 17, wherein the statistical analysis comprises a determination of a structural periodicity of α-keratin and collagen in the sample.

20. The system of claim 17, wherein the statistical analysis comprises a determination of a structural periodicity of one or more lipids in the sample.

21. The system of claim 17, wherein the statistical analysis comprises a determination of a structural periodicity of one or more of α-keratin, collagen, and a portion of an internal organ in the sample.

22. The system of claim 1, wherein the analysis module performs one or more machine learning processes selected from a supervised learning process, an unsupervised learning process, a semi-supervised learning process, a reinforcement learning process, and a deep learning process.

23. The system of claim 22, wherein the machine learning process comprises a deep learning process.

24. The system of claim 23, wherein the deep learning process comprises one of a convolutional neural network, a recurrent neural network, and a recurrent convolutional neural network.

25. The system of claim 22, wherein the machine learning process is trained using a training dataset comprising pathology lab image data, diffraction pattern data, subject data, or any combination thereof from one or more control samples.

26. The system of claim 25, wherein the training dataset is updated as new sample data are uploaded to the computer database.

27. The system of claim 1, wherein the subject-sample data further comprises subject data comprising one or more of a species, an age, a weight, a body condition score, a sex, ancestry data, genetic data, behavioral data of the subject.

28. The system of claim 1, wherein the objective-diagnostic indicator comprises an indicator of a likelihood that the sample indicates positive or negative probability for any of disease, cancer, and pathological abnormalities including cases caused by environmental exposure or heavy metal poisoning of the subject.

29. The system of claim 28, wherein the cancer is one of breast cancer, brain cancer, bone cancer, lung cancer, cervical cancer, bladder cancer, head and neck cancer, kidney cancer, intestinal cancer, liver cancer, ovarian cancer, pancreatic cancer, prostate cancer, skin cancer, throat cancer, oral cancer, and vaginal cancer.

30. The system of claim 1, wherein the subject-sample data includes pathology lab image data that includes micrographs of stained in vitro tissue specimens.
